(19) United States
(12) Patent Application Publication
(10) Pub. No.: US 2015/0094221 A1
(43) Pub. Date: Apr. 2, 2015

(54) Method for Indicating the Presence or Non-Presence of Prostate Cancer

(71) Applicant: Phadia AB, Uppsala (SE)

(72) Inventors: Henrik Grönberg, Stockholm (SE); Martin Eklund, Stockholm (SE)

(21) Appl. No.: 14/400,298

(22) PCT Filed: May 16, 2013
(86) PCT No.: PCT/SE2013/050554

§ 371 Date: Nov. 10, 2014

(30) Foreign Application Priority Data
May 16, 2012 (SE) 1250508-7
Nov. 20, 2012 (SE) 1251309-9

(51) Int. Cl. C12Q 1/68; G01N 33/574

(52) U.S. Cl. CPC C12Q 1/6886; G01N 33/57434; C12Q 2600/156; G01N 2333/96441
USPC 506/9; 435/6.11; 506/16

(57) ABSTRACT

The present invention relates generally to the detection and identification of various forms of genetic markers, and various forms of proteins, which have the potential utility as diagnostic markers. By determining the level of a plurality of biomarkers and genetic markers in a patient sample, and combining the obtained values according to a predefined formula, it is possible to determine if it is likely that the patient suffers from prostate cancer.

### FIELD OF THE INVENTION

The present invention relates generally to the detection and identification of various forms of genetic markers, and various forms of proteins, which have the potential utility as diagnostic markers. In particular, the present invention relates to the simultaneous use of multiple diagnostic markers for improved detection of prostate cancer.

### BACKGROUND OF THE INVENTION

The measurement of serum prostate specific antigen (PSA) is widely used for the screening and early detection of prostate cancer (PCa). As discussed in the public report “Polygenic Risk Score Improves Prostate Cancer Risk Prediction: Results from the Stockholm-1 Cohort Study” by Markus Aly and co-authors as published in EUROPEAN UROLOGY 60 (2011) 21-28 (which is incorporated by reference herein), serum PSA that is measurable by current clinical immunoassays exists primarily as either the free “non-complexed” form (free PSA), or as a complex with a-lantichymotrypsin (ACT). The ratio of free to total PSA in serum has been demonstrated to significantly improve the detection of PCa. Other factors, like age and documented family history may also improve the detection of PCa further. The measurement of genetic markers related to PCa, in particular single nucleotide polymorphisms (SNP), is an emerging modality for the screening and early detection of prostate cancer. Analysis of multiple PCa related SNPs can, in combination with biomarkers like PSA and with general information about the patient improve the risk assessment through a combination of several SNPs into a genetic score.

The screening and early detection of prostate cancer is a complicated task, and to date no single biomarker has been proven sufficiently good for specific and sensitive mapping of the male population. Therefore, attempts have been spent on combining biomarker levels in order to produce a formula which performs better in the screening and early detection of PCa. The most common example is the regular PSA test, which in fact is an assessment of “free” PSA and “total” PSA. PSA exists as one “non-complex” form and one form where PSA is in complex formation with alpha-lantichymotrypsin. Another such example is the use of combinations of concentrations of free PSA, total PSA, and one or more pro-enzyme forms of PSA for the purpose of diagnosis, as described in WO03100079 (METHOD OF ANALYZING PROENZYME FORMS OF PROSTATE SPECIFIC ANTIGEN IN SERUM TO IMPROVE PROSTATE CANCER DETECTION) which is incorporated by reference herein. The one possible combination of PSA concentrations and pro-enzyme concentrations that may result in improved performance for the screening and early detection of PCa is the phi index. Phi was developed as a combination of PSA, free PSA, and a PSA precursor form [−2]proPSA to better detecting PCa for men with a borderline PSA test (e.g. PSA 2-10 ng/mL) and non-suspicious digital rectal examination, as disclosed in the report “Cost-effectiveness of Prostate Health Index for prostate cancer detection” by Nichol M B and co-authors as published in BJU Int. 2011 November 11. doi: 10.1111/j.1464-410X.2011.10751.x. which is incorporated by reference herein. Another such example is the combination of psp94 and PSA, as described in US2012021925 (DIAGNOSTIC ASSAYS FOR PROSTATE CANCER USING PSP94 AND PSA BIOMARKERS).

There are other biomarkers of potential diagnostic or prognostic value for assessing if a patient suffers from PCa, including MIC-1 as described in the report “Macrophage Inhibitory Cytokine 1: A New Prognostic Marker in Prostate Cancer” by David A. Brown and co-authors as published in Clin Cancer Res 2009; 15(21):OF1-7, which is incorporated by reference herein.

Attempts to combine information from multiple sources into one algorithmic model for the prediction of PCa risk has been disclosed in the past. In the public report “Blood Biomarker Levels to Aid Discovery of Cancer-Related Single-Nucleotide Polymorphisms: Kallikreins and Prostate Cancer” by Robert Kleins and co-authors as published in Cancer Prev Res (2010), 3(5):611-619 (which is incorporated by reference herein), the authors discuss how blood biomarkers can aid the discovery of novel SNP, but also suggest that there is a potential role for incorporating both genotype and biomarker levels in predictive models. Furthermore, this report provides evidence that the non-additive combination of genetic markers and biomarkers in concert may have predictive value for the estimation of PCa risk. Later, Xu and co-inventors disclosed a method for correlating genetic markers with prostate cancer, primarily for the purpose of identifying subjects suitable for chemopreventive therapy using 5-alpha reductase inhibitor medication (e.g. dutasteride or finasteride) in the patent application WO2012031207 (which is incorporated by reference herein). In concert, these two public disclosures summarizes the prior art of combining genetic information and biomarker concentration for the purpose of estimating PCa risk.

The current performance of the PSA screening and early detection is approximately a sensitivity of 80% and specificity of 30%. It is estimated that approximately 65% will undergo unnecessary prostate biopsy and that 15-20% of the clinically relevant prostate cancers are missed in the current screening. In the United States alone, about 1 million biopsies are performed every year, which results in about 192 000 new cases being diagnosed. Hence, also a small improvement of diagnostic performance will result both in major savings in healthcare expenses due to fewer biopsies and in less human suffering from invasive diagnostic procedures.

The current clinical practice (in Sweden) is to use total PSA as biomarker for detection of asymptomatic and early prostate cancer. The general cutoff value for further evaluation with a prostate biopsy is 3 ng/mL. However, due to the negative consequences of PSA screening there is no organized PSA screening recommended in Europe or North America today.

Therefore, a need exists to develop assays for improving the detection and determination of early prostate cancer in a patient.

### SUMMARY OF THE INVENTION

The present invention is based on the discovery that the combination of diagnostic markers of different origin may improve the ability to detect PCa. In particular, the numbers of false positive results, i.e. patients without cancer who receive a positive diagnosis and are followed up with biopsy, are reduced. This can result not only in fewer men being subjected to the potential risks of invasive biopsy, but also results in major savings for the society, because unnecessary examinations can be avoided.

Accordingly, based on the discoveries of the present invention, a first aspect of the present invention provides a method for indicating a presence or non-presence of prostate cancer (PCa) in an individual, comprising the steps of:
1. Obtaining at least one biological sample from said individual;
2. In said biological sample, determining
   a) a presence or concentration of at least one PCa related biomarker;
   b) a PCa related genetic status of said individual by determining a presence of at least one SNP related to PCa; and
   c) a PCa biomarker concentration related genetic status of said individual by determining a presence of at least one SNP related to a PCa biomarker concentration;
3. Combining data from said individual regarding said presence or concentration of at least one PCa related biomarker, data from said individual regarding said PCa related genetic status, and data from said individual regarding PCa biomarker concentration related genetic status, to form an overall composite value;
4. Correlating said overall composite value to the presence of PCa in said individual by comparing the overall composite value to a pre-determined cut-off value established with control samples of known PCa and benign disease diagnosis;

wherein the presence or concentration of at least one of the biomarkers (i) PSA, (ii) total PSA (tPSA), (iii) intact PSA (iPSA), (iv) free PSA (fPSA), and (v) HK2, is determined and included in the overall composite value.

In an embodiment of the method according to the first aspect above, the presence or concentration of at least two, preferably at least three, more preferably at least four, of the biomarkers (i) PSA, (ii) total PSA (tPSA), (iii) intact PSA (iPSA), (iv) free PSA (fPSA), and (v) HK2, is determined and included in the overall composite value. In this regard, any combination of the above-listed biomarkers may be determined and included in the overall composite value.

According to an embodiment of the invention according to the first aspect above, one or more of the method steps, typically steps 3 and/or 4 are provided by means of a non-transitory computer-readable medium when executed in a computer comprising a processor and memory.

A second aspect of the invention provides a method for indicating a presence or non-presence of prostate cancer (PCa) in an individual, comprising the steps of:
1. Providing at least one biological sample from said individual;
2. In said biological sample, determining
   a) a presence or concentration of at least two PCa related biomarkers; and
   b) a PCa related genetic status of said individual by determining a presence of at least one SNP related to PCa; and
3. Combining data from said individual regarding said presence or concentration of at least two PCa related biomarkers, to form a biomarker composite value representing the PCa biomarker-related risk of developing PCa;
4. Combining data from said individual regarding said PCa related genetic status, to form a genetics composite value representing the genetics-related risk of developing PCa;
5. Combining the biomarker composite value and the genetics composite value to form an overall composite value to predict the presence of PCa in said individual by comparing said overall composite value to a pre-determined cut-off value established with control samples of known PCa and benign disease diagnosis;

wherein the presence or concentration of at least one and at most four of the biomarkers (i) PSA, (ii) total PSA (tPSA), (iii) intact PSA (iPSA), (iv) free PSA (fPSA), and (v) HK2, is determined and included in the biomarker composite value.

In an embodiment of the method according to the second aspect above, the presence or concentration of at least one and at most three, such as at most two of the biomarkers (i) PSA, (ii) total PSA (tPSA), (iii) intact PSA (iPSA), (iv) free PSA (fPSA), and (v) HK2, is determined and included in the biomarker composite value. In this regard, any combination of the above-listed biomarkers may be determined and included in the biomarker composite value.

In an embodiment according to the second aspect above, the method further comprises a step 2 c) determining, in said biological sample, a PCa biomarker concentration related genetic status of said individual by determining a presence of at least one SNP related to a PCa biomarker concentration;

and step 4 comprises combining data from said individual regarding said PCa related genetic status and said PCa biomarker concentration related genetic status, to form a genetics composite value representing the genetics-related risk of developing PCa.

According to an embodiment of the invention according to the second aspect above, one or more of the method steps, typically steps 3 and/or 4 and/or 5, are provided by means of a non-transitory computer-readable medium when executed in a computer comprising a processor and memory.

In an embodiment of the first or second aspect of the present invention, the SNP related to PCa includes at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197.

In an embodiment of the first or second aspect of the present invention, the SNP related to a PCa biomarker concentration includes at least one of rs3213764, rs1354774, and rs1227732.

In an embodiment of the first or second aspect of the present invention, the method further comprises determining a Body Mass Index (BMI) related genetic status of said individual by determining a presence of at least one SNP related to the BMI, and wherein data from said individual regarding said SNP related to the BMI are included in the combined data forming said overall composite value.

In an embodiment of the first or second aspect, the SNP related to the BMI of said individual includes at least one of rs3817334, rs10767664, rs2241423, rs7359397, rs7190603, rs571312, rs29941, rs2287019, rs2815752, rs713586, rs2867125, rs9816226, rs10938397, and rs1558902.

In an embodiment of the method according to the first or second aspect, at least one of the genetic markers listed in Table 1 is determined.

In another embodiment of the first or second aspect of the invention, the method further comprises collecting the family history regarding PCa and physical data from said individual, and wherein said family history and/or physical data are included in the combined data forming said overall composite value.

In an embodiment of the method according to the first or second aspect, the presence or concentration of MIC-1 and/or MSMB is further determined, and included either in the biomarker composite value or directly in the overall composite value.

In an embodiment of the first or second aspect, the biological sample is a blood sample.

In an embodiment of the first or second aspect of the invention, the overall composite value and/or the biomarker composite value and/or the genetics composite value is calculated using a method in which the non-additive effect of a SNP related to a PCa biomarker concentration and the corresponding PCa biomarker concentration is utilized.

In an embodiment of the method according to the first or second aspect, the determination of the genetic status is conducted by use of MALDI mass spectrometry.

In an embodiment of the method of the first or second aspect, the determination of a presence or concentration of said PCa biomarkers is conducted by use of microarray technology.

A third aspect of the present invention provides an assay device for performing step 2 of the method according to the first or second aspect as described above.

In an embodiment of the third aspect, an assay device is provided for performing step 2a (i.e. determining a presence or concentration of at least one PCa related biomarker), step 2b (i.e. determining a PCa related genetic status of said individual by determining a presence of at least one SNP related to PCa), and step 2c (i.e. determining a PCa biomarker concentration related genetic status of said individual by determining a presence of at least one SNP related to a PCa biomarker concentration) of the above-described method for indicating a presence or non-presence of prostate cancer in an individual, according to the first aspect of the invention as described above, said assay device comprising a solid phase having immobilised thereon at least three different types of ligands, wherein:
the first type of said ligands comprises at least one ligand, which binds specifically to a biomarker related to PCa, such as at least one of PSA, iPSA, tPSA, fPSA, and hK2, and optionally also MIC-1 and/or MSMB;
the second type of said ligands comprises at least one ligand, which binds specifically to a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197; and
the third type of said ligands comprises at least one ligand, which binds specifically to a SNP related to a PCa biomarker concentration, such as at least one of rs3213764, rs1354774 and rs1227732.

In another embodiment of the third aspect, an assay device is provided for performing step 2a (i.e. determining a presence or concentration of at least one PCa related biomarker), and step 2b (i.e. determining a PCa related genetic status of said individual by determining a presence of at least one SNP related to PCa) of the above-described method for indicating a presence or non-presence of prostate cancer in an individual, according to the second aspect of the invention as described above, said assay device comprising a solid phase having immobilised thereon at least two different types of ligands, wherein:
the first type of said ligands comprises at least two different ligands, each of which binding specifically to a biomarker related to PCa, such as one of PSA, iPSA, tPSA, fPSA, and hK2, and optionally also MIC-1 and/or MSMB; and
the second type of said ligands comprises at least one ligand, which binds specifically to a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197.

This embodiment may further include that said assay device for performing step 2a and step 2b of the method according to the second aspect further is adapted for performing step 2c of the method according to the second aspect, in which case the solid phase further has a third type of ligand immobilised, wherein said third type of ligand comprises at least one ligand, which binds specifically to a SNP related to a PCa biomarker concentration, such as at least one of rs3213764, rs1354774 and rs1227732.

In an embodiment according to the third aspect, the assay device is also suitable for determining a BMI related genetic status, in which case the solid phase further has a fourth type of ligand immobilised, wherein said fourth type of ligand comprises at least one ligand, which binds specifically to a SNP related to the BMI, such as at least one of rs3817334, rs10767664, rs2241423, rs7359397, rs7190603, rs571312, rs29941, rs2287019, rs2815752, rs713586, rs2867125, rs9816226, rs10938397, and rs1558902.

In an embodiment, the solid phase of the assay device may comprise one or several separate structures, each of said structures having a flat form, such as a microtiter plate or a microarray chip, or a bead-like form.

According to a fourth aspect of the invention, a test kit is provided for performing step 2 of the method according to the first or second aspect as described above.

In an embodiment of the fourth aspect, a test kit is provided for performing step 2a (i.e. determining a presence or concentration of at least one PCa related biomarker), step 2b (i.e. determining a PCa related genetic status of said individual by determining a presence of at least one SNP related to PCa), and step 2c (i.e. determining a PCa biomarker concentration related genetic status of said individual by determining a presence of at least one SNP related to a biomarker concentration) of the above-described method for indicating a presence or non-presence of prostate cancer in an individual, according to the first aspect of the invention as described above, comprising a corresponding assay device as described above and at least three different types of detection molecules, wherein:
the first type of said detection molecules comprises at least one detection molecule, which is capable of detecting a biomarker related to PCa, such as at least one of PSA, iPSA, tPSA, fPSA, and hK2, and optionally also MIC-1 and/or MSMB;
the second type of said detection molecules comprises at least one detection molecule, which is capable of detecting a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197; and
the third type of said detection molecules comprises at least one detection molecule, which is capable of detecting a SNP related to a PCa biomarker concentration, such as at least one of rs3213764, rs1354774 and rs1227732.

In another embodiment of the fourth aspect, a test kit is provided for performing step 2a (i.e. determining a presence or concentration of at least one PCa related biomarker), and step 2b (i.e. determining a PCa related genetic status of said individual by determining a presence of at least one SNP related to PCa) of the above-described method for indicating a presence or non-presence of prostate cancer in an individual, according to the second aspect above, comprising a corresponding assay device as described above and at least two different types of detection molecules, wherein:
the first type of said detection molecules comprises at least two different detection molecule, each of which is capable of detecting a biomarker related to PCa, such as at least one of PSA, iPSA, tPSA, fPSA, and hK2, and optionally also MIC-1 and/or MSMB, provided that said at least two different detection molecules are capable of detecting different biomarkers related to PCa; and
the second of said detection molecules comprises at least one detection molecule, which is capable of detecting a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197.

This embodiment may further include that said test kit for performing step 2a and step 2b of the method according to the second aspect is also adapted for performing step 2c of the method according to the second aspect, in which case the test kit comprises a corresponding assay device as described above and a third type of detection molecule, wherein said third type of detection molecule comprises at least one detection molecule, which binds specifically to a SNP related to a PCa biomarker concentration, such as at least one of rs3213764, rs1354774 and rs1227732.

In an embodiment of the fourth aspect, the test kit comprises an assay device that is further suitable for determining a BMI related genetic status, and a fourth type of detection molecule, wherein said fourth type of detection molecule comprises at least one detection molecule, which is capable of detecting a SNP related to the BMI, such as at least one of rs3817334, rs10767664, rs2241423, rs7359397, rs7190603, rs571312, rs29941, rs2287019, rs2815752, rs713586, rs2867125, rs9816226, rs10938397, and rs1558902.

In an embodiment of any one of the aspects relating to a test kit as described above, each type of detection molecule (i.e. the first, second, third and/or fourth type of detection molecule) may comprise at least two different detection molecules, provided that said at least two different detection molecules are capable of detecting 1) different biomarkers related to PCa (first type), or 2) different SNPs related to PCa (second type), or 3) different SNPs related to a PCa biomarker concentration (third type), or 4) different SNPs related to the BMI.

A fifth aspect of the present invention provides an assay device comprising a solid phase having immobilised thereon at least three different types of ligands, wherein:
the first type of said ligands comprises at least one ligand, which binds specifically to a biomarker related to PCa, selected from at least one of PSA, iPSA, tPSA, fPSA, and hK2, and optionally also MIC-1 and/or MSMB;
the second type of said ligands comprises at least one ligand, which binds specifically to a SNP related to PCa, selected from at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197; and
the third type of said ligands comprises at least one ligand, which binds specifically to a SNP related to a PCa biomarker concentration, selected from at least one of rs3213764, rs1354774 and rs1227732.

A sixth aspect provides an assay device comprising a solid phase having immobilised thereon at least two different types of ligands, wherein:
the first type of said ligands comprises at least two ligands, each of which binding specifically to a biomarker related to PCa, selected from at least one of PSA, iPSA, tPSA, fPSA, and hK2, and optionally also MIC-1 and/or MSMB; and
the second type of said ligands comprises at least one ligand, which binds specifically to a SNP related to PCa, selected from at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197.

In an embodiment of the assay device according to the sixth aspect, the solid phase further has a third type of ligand, wherein the third type of ligand comprises at least one ligand, which binds specifically to a SNP related to a PCa biomarker concentration, selected from at least one of rs3213764, rs1354774 and rs1227732.

In an embodiment of the assay device according to the fifth or sixth aspect, the solid phase further has a fourth type of ligand immobilised, wherein said fourth type of ligand comprises at least one ligand, which binds specifically to a SNP related to the BMI, selected from at least one of rs3817334, rs10767664, rs2241423, rs7359397, rs7190603, rs571312, rs29941, rs2287019, rs2815752, rs713586, rs2867125, rs9816226, rs10938397, and rs1558902.

A seventh aspect of the invention provides a non-transitory computer readable medium comprising instructions for causing a computer to perform steps of the above-described method for indicating a presence or non-presence of prostate cancer in an individual in accordance with the first aspect of the invention; such as to perform at least step 3 (i.e. combining data from said individual regarding said presence or concentration of at least one PCa related biomarker, and data from said individual regarding PCa related genetic status to form an overall composite value) and step 4 (correlating said overall composite value to the presence of PCa in said individual by comparing the overall composite value to a pre-determined cut-off value established with control samples of known PCa and benign disease diagnosis) of said method; such as step 1 (i.e. obtaining at least one biological sample from said individual), steps 2a, 2b, and 2c (in the biological sample, determining a presence or concentration of at least one PCa related biomarker, a PCa related genetic status of said individual by determining a presence of at least one SNP related to PCa, and a PCa biomarker concentration related genetic status of said individual by determining a presence of at least one SNP related to a PCa biomarker concentration), step 3 and step 4 of said method.

An eighth aspect provides a non-transitory computer readable medium comprising instructions for causing a computer to perform steps of the above-described method for indicating a presence or non-presence of prostate cancer in an individual in accordance with the second aspect of the invention; such as to perform at least step 3 (i.e. combining data from said individual regarding said presence or concentration of at least two PCa related biomarkers, to form a biomarker composite value representing the PCa biomarker-related risk of developing PCa) and step 4 (i.e. combining data from said individual regarding said genetic status, to form a genetics composite value representing the genetics-related risk of developing PCa) and/or step 5 (i.e. combining the biomarker composite value and the genetics composite value to form an overall composite value to predict the presence of PCa in said individual by comparing said overall composite value to a pre-determined cut-off value established with control samples of known PCa and benign disease diagnosis) of said method; such as step 1 (i.e. obtaining at least one biological sample from said individual), steps 2a and 2b (in the biological sample, determining a presence or concentration of at least two PCa related biomarkers, and a PCa related genetic status of said individual by determining a presence of at least one SNP related to PCa), step 3, step 4, and optionally also step 5 of said method.

An embodiment of the eighth aspect further comprises instructions for causing a computer to perform step 2c of the method according to the second aspect (in the biological sample, determining a PCa biomarker concentration related genetic status of said individual by determining a presence of at least one SNP related to a PCa biomarker concentration).

In an embodiment of the seventh or eighth aspect, the non-transitory computer readable medium further comprises instructions, such as software code means, for determining a BMI related genetic status of an individual by determining a presence of at least one SNP related to the BMI.

A ninth aspect of the invention provides an apparatus comprising an assay device as described above and a corresponding non-transitory computer readable medium as described above.

### BRIEF DESCRIPTION OF THE DRAWINGS

FIG. 1 shows the Receiver Operating Characteristic (ROC) curve of two different diagnostic models for assessing if an individual is suffering from PCa.

FIG. 2 shows the ROC curves for two different diagnostic models for assessing if an individual is suffering from PCa, both alone and supplemented with information from genetic markers (SNPs).

FIG. 3 shows the ROC curves for the linear model of Example 2 illustrating the difference in performance between PSA (301) and the multiparametric model (302) in prediction of PCa.

FIG. 4 shows an example of a decision tree to predict whether a subject should be referred to biopsy.

FIG. 5 shows six correlation plots of protein biomarker levels from 450 individuals.

### DETAILED DESCRIPTION OF THE INVENTION

For the purpose of this application and for clarity, the following definitions are made:

The term “PSA” refers to serum prostate specific antigen in general. PSA exists in different forms, where the term “free PSA” refers to PSA that is unbound or not bound to another molecule, the term “bound PSA” refers to PSA that is bound to another molecule, and finally the term “total PSA” refers to the sum of free PSA and bound PSA. The term “F/T PSA” is the ratio of unbound PSA to total PSA. There are also molecular derivatives of PSA, where the term “proPSA” refers to a precursor inactive form of PSA and “intact PSA” refers to an additional form of proPSA that is found intact and inactive.

The term “diagnostic assay” refers to the detection of the presence or nature of a pathologic condition. It may be used interchangeably with “diagnostic method”. Diagnostic assays differ in their sensitivity and specificity.

One measure of the usefulness of a diagnostic tool is “area under the receiver-operator characteristic curve”, which is commonly known as ROC-AUC statistics. This widely accepted measure takes into account both the sensitivity and specificity of the tool. The ROC-AUC measure typically ranges from 0.5 to 1.0, where a value of 0.5 indicates the tool has no diagnostic value and a value of 1.0 indicates the tool has 100% sensitivity and 100% specificity.

The term “sensitivity” refers to the proportion of all subjects with PCa that are correctly identified as such (which is equal to the number of true positives divided by the sum of the number of true positives and false negatives).

The term “specificity” refers to the proportion of all subjects healthy with respect to PCa (i.e. not having PCa) that are correctly identified as such (which is equal to the number of true negatives divided by the sum of the number of true negatives and false positives).

The term biomarker refers to a protein, a part of a protein, a peptide or a polypeptide, which may be used as a biological marker, e.g. for diagnostic purposes.

The term single nucleotide polymorphisms (SNP) refer to the genetic properties of a defined locus in the genetic code of an individual. An SNP can be related to increased risk for PCA, and can hence be used for diagnostic or prognostic assessments of an individual. The Single Nucleotide Polymorphism Database (dbSNP) is an archive for genetic variation within and across different species developed and hosted by the National Center for Biotechnology Information (NCBI) in collaboration with the National Human Genome Research Institute (NHGRI), both located in the US. Although the name of the database implies a collection of one class of polymorphisms only (i.e., single nucleotide polymorphisms (SNP)), it in fact contains a range of molecular variation. Every unique submitted SNP record receives a reference SNP ID number (“rs#”; “refSNP cluster”). In the present application, SNP are mainly identified using rs# numbers.

The term “ligand” refers to a molecule attached or immobilised to a solid support, optionally via a linker molecule, for the purpose of binding a sought-after molecule to the solid support. As a non-limiting example, a ligand can be an antibody attached to a support, said antibody being capable of binding the sought-after molecule. As another non-limiting example, a ligand can be a nucleic acid capable of binding a sought-after molecule (typically the complementary nucleic acid). As yet another non-limiting example, a ligand can be a small synthetic molecule capable of binding a sought-after molecule.

The present invention provides diagnostic methods to aid in detecting and/or determining the presence of prostate cancer in a subject, with the explicit purpose of reducing the number of false positive results. False positive results are expensive both in respect to the cost of unnecessary treatment and in the respect of unnecessary human suffering. The basic principle of the invention is the use of combinations of biomarkers and genetic information in such a manner that the combinatorial use of the assessed information about the individual improves the quality of the diagnosis.
Collecting the family history regarding PCa from a patient (Category HIST).
Collecting patient physical data, such as weight, BMI, age and similar (Category PPD)
Obtaining a number of biological samples from said patient.
In said biological samples, quantifying the presence or concentration of a plurality of defined biomarkers (Category Biomarker).
In said biological samples, quantifying the genetic status of said patients with respect to a plurality of defined SNPs related to PCa (Category SNPpc).
In said biological samples, quantifying the genetic status of said patients with respect to a plurality of defined SNPs related to biomarker expression levels (Category SNPbm).
Combining data from at least three, such as four or all five categories as defined above to form an overall composite value for the use in the detection of early prostate cancer.
Determining by using said overall composite value, alone or in combination with further data, if the patient is likely to suffer from PCa.

In more detail, the step comprising the collection of family history includes, but is not limited to, the identification of if any closely related male family member (such as the father, brother or son of the patient) suffers or have suffered from PCa.

Physical information regarding the patient is typically obtained through a regular physical examination wherein age, weight, height, BMI and similar physical data are collected.

Collecting biological samples from a patient includes, but is not limited to plasma, serum, DNA from peripheral white blood cells and urine.

The quantification of presence or concentration of biomarkers in a biological sample can be made in many different ways. One common method is the use of enzyme linked immunosorbent assays (ELISA) which uses antibodies and a calibration curve to assess the presence and (where possible) the concentration of a selected biomarker. ELISA assays are common and known in the art, as evident from the publication “Association between saliva PSA and serum PSA in conditions with prostate adenocarcinoma.” by Shiiki N and co-authors, published in Biomarkers. 2011 September; 16(6):498-503, which is incorporated by reference herein. Another common method is the use of a microarray assay for the quantification of presence or concentration of biomarkers in a biological sample. A typical microarray assay comprises a flat glass slide onto which a plurality of different capture reagents (typically an antibody) each selected to specifically capture one type of biomarker is attached in non-overlapping areas on one side of the slide. The biological sample is allowed to contact, for a defined period of time, the area where said capture reagents are located, followed by washing the area of capture reagents. At this point, in case the sought-after biomarker was present in the biological sample, the corresponding capture reagent will have captured a fraction of the sought-after biomarker and keep it attached to the glass slide also after the wash. Next, a set of detection reagents are added to the area of capture reagents (which now potentially holds biomarkers bound), said detection reagents being capable of (i) binding to the biomarker as presented on the glass slide and (ii) producing a detectable signal (normally through conjugation to a fluorescent dye). It is typically required that one detection reagent per biomarker is added to the glass slide. There are many other methods capable of quantifying the presence or concentration of a biomarker, including, but not limited to, immunoprecipitation assays, immunofluorescense assays, radio-immuno-assays, and mass spectrometry using matrix-assisted laser desorption/ionization (MALDI), to mention a few examples.

The quantification of genetic status through the analysis of a biological sample typically involves MALDI mass spectrometry analysis based on allele-specific primer extensions, even though other methods are equally applicable. This applies to any type of genetic status, i.e. both SNPs related to PCa and SNPs related to biomarker expression.

The combination of data can be any kind of algorithmic combination of results, such as a linear combination of data wherein the linear combination improves the diagnostic performance (for example as measured using ROC-AUC). Another possible combination includes a non-linear polynomial relationship.

Suitable biomarkers for diagnosing PCa include, but are not limited to, Prostate-specific antigen (PSA) in either free form or complexed form, pro PSA (a collection of isoforms of PSA) and in particular the truncated form (−2) pro PSA, human prostatic acid phosphatase (PAP), human kallikrein 2 (hK2), early prostate cancer antigen (EPCA), Prostate Secretory Protein (PSP94; also known as beta-microseminoprotein and MSMB), glutathione S-transferase π (GSTP1), and α-methylacyl coenzyme A racemase (AMACR). Related biomarkers, which may be useful for improving the diagnostic accuracy of the method includes Macrophage Inhibitory Cytokine 1 (MIC-1; also known as GDF15).

Suitable SNPs related to PCa include, but are not limited to rs12621278 (Chromosome 2, locus 2q31.1), rs9364554 (Chromosome 6, locus 6q25.3), rs10486567 (Chromosome 7, locus 7p15.2), rs6465657 (Chromosome 7, locus 7q21.3), rs2928679 (Chromosome 8, locus 8p21), rs6983561 (Chromosome 8, locus 8q24.21), rs16901979 (Chromosome 8, locus 8q24.21), rs16902094 (Chromosome 8, locus 8q24.21), rs12418451 (Chromosome 11, locus 11q13.2), rs4430796 (Chromosome 17, locus 17q12), rs11649743 (Chromosome 17, locus 17q12), rs2735839 (Chromosome 19, locus 19q13.33), rs9623117 (Chromosome 22, locus 22q13.1), and rs138213197 (Chromosome 17, locus 17q21).

Suitable SNPs related to PCa further include, but are not limited to rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, and rs2405942.

Suitable SNPs related to PCa further include, but are not limited to rs138213197 as described in the report “Germline mutations in HOXB13 and prostate-cancer risk.” by Ewing C M and co-authors as published in N Engl J Med. 2012 January 12; 366(2):141-9 (which is incorporated by reference herein), 1100delC (22q12.1) and I157T (22q12.1) as described in the report “A novel founder CHEK2 mutation is associated with increased prostate cancer risk.” by Cybulski C and co-authors as published in Cancer Res. 2004 April 15; 64(8):2677-9 (which is incorporated by reference herein), and 657del5 (8q21) as described in the report “NBS1 is a prostate cancer susceptibility gene” by Cybulski C and co-authors as published in Cancer Res. 2004 February 15; 64(4):1215-9 (which is incorporated by reference herein).

Suitable SNPs related to other processes than PCa include, but are not limited to rs3213764, rs1354774, rs2736098, rs401681, rs10788160 rs11067228, all being related to the expression level of PSA.

Suitable SNPs related to other processes than PCa further include, but are not limited to rs1363120, rs888663, rs1227732, rs1054564, all being related to the expression level of the inflammation cytokine biomarker MIC1.

Suitable SNPs related to other processes than PCa further include, but are not limited to rs3817334, rs10767664, rs2241423, rs7359397, rs7190603, rs571312, rs29941, rs2287019, rs2815752, rs713586, rs2867125, rs9816226, rs10938397, and rs1558902 all being related to the Body Mass Index (BMI) of an individual.

As has been discussed previously, the assessment of the performance of PCa screening efficiency is difficult. Although the ROC-AUC characteristics provide some insight regarding performance, additional methods are desirable. One alternative method for assessing performance of PCa screening is to calculate the percentage of positive biopsies at a given sensitivity level and compare the performance of screening using PSA alone with any novel method for screening. This however requires that the performance of PSA is accurately defined.

One example of an assessment performance of PSA screening has been disclosed by IM Thompson and co-authors in the report “Assessing prostate cancer risk: results from the Prostate Cancer Prevention Trial.” as published in J Natl Cancer Inst. 2006 April 19; 98(8):529-34 (which is incorporated by reference herein). In this report, prostate biopsy data from men who participated in the Prostate Cancer Prevention Trial (PCPT) was used to determine the sensitivity of PSA. In total, 5519 men from the placebo group of the PCPT who underwent prostate biopsy, had at least one PSA measurement and a digital rectal examination (DRE) performed during the year before the biopsy, and had at least two PSA measurements performed during the 3 years before the prostate biopsy was included. This report discloses that when using a PSA value of 3 ng/mL as a cutoff about 41% of the high-grade cancers (i.e. cancers with Gleason score 7 or above) will be missed.

A second analysis using the same study population has been disclosed by IM Thompson and co-authors in “Operating characteristics of prostate-specific antigen in men with an initial PSA level of 3.0 ng/ml or lower” as published in JAMA. 2005 July 6; 294(1):66-70 (which is incorporated by reference herein). In this report, the authors present an estimate of the sensitivity and specificity of PSA for all prostate cancer, Gleason 7+ and Gleason 8+. When using 3.1 ng/mL as PSA cut off value for biopsy a sensitivity of 56.7% and a specificity of 82.3% for Gleason 7+ tumors was estimated. In this report the authors concluded that there is no cut point of PSA with simultaneous high sensitivity and high specificity for monitoring healthy men for prostate cancer, but rather a continuum of prostate cancer risk at all values of PSA. This illustrates the complication with PSA as a screening test while still acknowledging the connection of PSA with prostate cancer.

One inevitable consequence of the difficulties in obtaining accurate and comparable estimates of the predictive performance of any given diagnostic or prognostic model in the screening of PCa is that when calculating the relative improvement of a novel method as compared to using PSA alone, the calculated relative improvement will vary depending on many factors. One important factor that influences the calculated relative improvement is how the control group (i.e. known negatives) is obtained. Since it is unethical to conduct biopsies on subjects where there are no indications of PCa, the control group will be selected with bias. Thus, the relative improvement of a novel method will depend on how the control group was selected, and there are multiple fair known methods to select control groups. Any reported estimated improvement must therefore be seen in the light of such variance. To the best of our experience, we estimate that if the relative improvement of a novel method is reported to be 15% as compared to the PSA value alone using one fair known method for selecting the control group, said novel method would be at least 10% better than the PSA value alone using any other fair known method for selecting the control group.

To become used in a widespread manner in society, the performance of a screen must meet reasonable health economic advantages. A rough estimate is that a screening method performing about 15% better than PSA (i.e. avoiding 15% of the unnecessary biopsies) at the same sensitivity level, i.e. detecting the same number of prostate cancers in the population, would have a chance of being used in a widespread manner in the current cost level of public health systems. It is noted that even though significant efforts have been put on finding a combined model for the estimation of PCa risk (as exemplified in several of the cited documents in this patent application), no such combined method is currently in regular use in Europe. Thus, previous known multiparametric methods do not meet the socioeconomic standards to be useful in modern health care. The method of the current invention has better performance than previously presented combined methods and meet the socioeconomic performance requirements to at all be considered by a health care system.

One possible method for obtaining a screening method for PCa meeting the requirements for widespread use is to combine information from multiple sources. From an overview level, this comprises combining values obtained from biomarker analysis (e.g. PSA values), genetic profiles (e.g. the SNP profile), family history, and other sources. The combination as such has the possibility to produce a better diagnostic statement than any of the included factors alone. Attempts to combine values into a multiparametric model to produce better diagnostic statements have been disclosed in the past, as described elsewhere in the current application.

The combination of data can be any kind of algorithmic combination of results, such as a linear combination of data wherein the linear combination improves the diagnostic performance (for example as measured using ROC-AUC). Other possible methods for combining into a model capable of producing a diagnostic estimate include (but are not limited to) non-linear polynomials, support vector machines, neural network classifiers, discriminant analysis, random forest, gradient boosting, partial least squares, ridge regression, lasso, elastic nets, k-nearest neighbors. Furthermore, the book “The Elements of Statistical Learning: Data Mining, Inference, and Prediction, Second Edition” by T Hastie, R Tibshirani and J Friedman as published by Springer Series in Statistics, ISBN 978-0387848570 (which is incorporated by reference herein) describes many suitable methods for combining data in order to predict or classify a particular outcome.

The algorithm which turns the data from the three, four or five categories into a single value being indicative of if the patient is likely to suffer from PCa is preferably a non-linear function, wherein the dependency of different categories is employed for further increasing the diagnostic performance of the method. For example, one important dependency is the measured level of a selected biomarker combined with any associated genetic marker related to the expected expression level of said biomarker. In cases where an elevated concentration of the biomarker is found in a patient sample, and at the same time said patient is genetically predisposed of having lower levels of said biomarkers, the importance of the elevated biomarker level is increased. Likewise, if a biomarker level is clearly lower than normal in a patient being genetically predisposed to have high levels of said biomarkers, the contradictory finding increases the importance of the biomarker level interpretation.

The algorithm used for predicting PCa risk may benefit from using transformed variables, for example by using the log 10(PSA) value. Transformation is particularly beneficial for variables with a distribution that is deviating clearly from the normal distribution. Possible variable transformations include, but are not limited to, logarithm, inverse, square, and square root. It is further common to center each variable to zero average and unit variance.

When applied in practice, it will occasionally happen that one or a few measurements fail due to for example unforeseen technical problems, human error, or any other unexpected and uncommon reason. In such cases the data set obtained for an individual will be incomplete. Typically, such an incomplete data set would be difficult or even impossible to evaluate. However, the current invention relies on measurements of a large number of features of which many are partially redundant. This means that also for individuals for which the data set is incomplete, it will in many cases be possible to produce a high-quality assessment according to the invention. This is particularly true within categories, where for example the Kallikrein protein biomarkers (including but not limited to PSA and HK2) are correlated and partially redundant. Technically, it is therefore possible to apply an algorithmic two-step approach, wherein the kallikrein biomarker contribution is summarized into a kallikrein score. This kallikrein score is then in a second step being combined with other data (such as genetic score, age, and family history to mention a few non-limiting examples) to produce a diagnostic or prognostic statement on PCa. Similar two-step procedures can be implemented for other classes of biomarkers, such as genetic markers related to BMI or protein biomarkers related to MIC-1, to mention two non-limiting examples.

Genetic risk scores are also insensitive to small losses of data due to for example unforeseen technical problems, human error, or any other unexpected and uncommon reason. This is not due to redundancy because the contribution of one SNP to the risk score is typically not correlated to any other SNP. In the case of SNP, the risk change due to each SNP is small, and only by using multiple SNP related to a condition in concert, the risk change for said condition becomes large enough for having an impact on the model performance. The preferred number of SNP to form a genetic score is at least 3 SNP, even more preferably 10 SNP, yet even more preferably 25 SNP, still even more preferably 50 SNP, yet even more preferably 100 SNP, and still even more preferably 300 SNP. This means that the impact of any single SNP on the total result is typically small, and the omission of a few SNP will typically not alter the overall genetic score risk assessment in any large manner. Currently, the typical data loss in the large scale genetic measurements is on the order of 1-2%, meaning that if a genetic score is composed of 100 different SNP, the typical genetic characterization of an individual would provide information about 98-99 of these SNP's. The model as such can however withstand a larger loss in data, such as 5-7% loss of information, as illustrated in Example 4.

The redundancy aspect of the models for predicting PCa risk has important clinical consequences. It is known that measurements of biomarkers or genetic markers are sometimes failing and the process of retesting may take time if at all possible. Still, when applying the present invention, a high quality assessment of the PCa risk may be possible for individuals for which partial biomarker and genetic information is missing resulting in that a greater fraction of the individuals suitable for PCa risk assessment will indeed get the risk assessed. This results in less suffering for the individuals and reduces the cost for the society in that retests need not necessarily be conducted. For example, it is with the current invention possible to assess the risk for individuals with one or two kallikrein biomarker values missing in combination with a 5% of genetic information missing.

The redundancy aspect can be embodied in many different manners. One possible way to implement the redundancy aspect is to define a set of biomarkers representing biomarkers related to a common field or family. One non-limiting example of such a field or family is Kallikrein-like biomarkers. More than one defined set of biomarkers can be determined, and in addition still other biomarkers can be applied outside such a set. Typically, the sets are non-overlapping, i.e. any defined biomarker is only member of one defined set or used in a solitary manner. Next, for all biomarkers an attempt to determine a presence or concentration is made. In most cases the determination for all biomarkers will succeed, but occasionally one or a few values will be missing. To induce model robustness to missing values, it is possible to define a biomarker set composite value which can be determined using all or a subset of the members of the defined set. To work in practice, this requires that the members of the defined set of biomarkers are at least partially redundant. In the next step, the biomarker set composite value is combined with other biomarker values, other biomarker set composite values (if two or more sets of biomarkers were defined), genetic score related to PCa risk, genetic score related to other features (such as BMI or biomarker concentration, to mention two non-limiting examples), family history, age, and other information carriers related to PCa risk into an overall composite value. The overall composite value is finally used for the estimation of PCa risk.

The purpose of the biomarker set composite value is hence to serve as an intermediate value which can be estimated using incomplete data. Assume that a defined set of biomarker comprises N different biomarkers denoted B1, B2, B3, . . . BN, all related to the biomarker family B. In that case, there could be N different models available for calculating the family B biomarker composite value C:

$$C = f 1 ( B 1 , B 2 , B 3 , … BN )$$

$$C = f 2 ( B 2 , B 3 , … BN )$$

$$C = f 3 ( B 1 , B 3 , … BN )$$

$$…$$

$$C = fN ( B 1 , B 2 , B 3 , … BN - 1 )$$

Wherein f1( ), f2( ) . . . fN( ) are mathematical functions using the values for biomarkers B1, . . . BN as input and in some manner producing a single output C representing family B biomarker composite value. One non-limiting example of the functions f1( ) . . . fN( ) include linear combinations of the present arguments. With such a set of multiple functions capable of calculating C for all the cases of one single biomarker value missing, the calculation of the overall composite value becomes less sensitive to missing data. It is understood that the estimate of C might be of less good quality when not all data is present, but may still be good enough for use in the assessment of PCa risk. Thus, using such a strategy, only N−1 biomarker determinations have to succeed in order to produce an estimate of C. It is further possible to develop estimates for any number of lost data, i.e. if N−2 biomarker determinations have to succeed, another set of functions f( ) could be developed and applied to estimate C.

One suitable method for associating a SNP with a condition (for example PCa, or BMI>25, or elevated hk2 biomarker concentration in blood) has been described in the public report “Blood Biomarker Levels to Aid Discovery of Cancer-Related Single-Nucleotide Polymorphisms: Kallikreins and Prostate Cancer” by Robert Kleins and co-authors as published in Cancer Prev Res 2010; 3:611-619 (which is incorporated by reference herein). In this report, the authors describe how they could associate the SNP rs2735839 to elevated value of (free PSA)/(total PSA). Furthermore, they could associate the SNP rs10993994 to elevated PCa risk, elevated total PSA value, elevated free PSA value and elevated hk2 value, and finally SNP rs198977 was associated with elevated PCa risk, elevated value of (free PSA)/(total PSA), and elevated hk2 value.

One preferred method for combining information from multiple sources has been described in the public report “Polygenic Risk Score Improves Prostate Cancer Risk Prediction: Results from the Stockholm-1 Cohort Study” by Markus Aly and co-authors as published in EUROPEAN UROLOGY 60 (2011) 21-28 (which is incorporated by reference herein). Associations between each SNP and PCa at biopsy were assessed using a Cochran-Armitage trend test. Allelic odds ratios (OR) with 95% confidence intervals were computed using logistic regression models. For each patient, a genetic risk score was created by summing the number of risk alleles (0, 1, or 2) at each of the SNPs multiplied by the logarithm of that SNP's OR. Associations between PCa diagnosis and evaluated risk factors were explored in logistic regression analysis. The portion of the model related to non-genetic information included logarithmically transformed total PSA, the logarithmically transformed free-to-total PSA ratio, age at biopsy, and family history of PCa (yes or no). A repeated 10-fold cross-validation was used to estimate the predicted probabilities of PCa at biopsy. Ninety-five percent confidence intervals for the ROC-AUC values were constructed using a normal approximation. All reported p values were based on two-sided hypotheses.

### Example 1

To illustrate the current invention, a data set comprising 500 cases (subjects known to suffer from PCa) and 500 controls (subjects known not to suffer from PCa) from the Cancer of the Prostate Sweden (CAPS) data set was extracted. The CAPS data set has been discussed in the public domain, as evident in the report “A comprehensive association study for genes in inflammation pathway provides support for their roles in prostate cancer risk in the CAPS study.” by Zheng and co-authors as published in Prostate, 2006 October 1; 66(14):1556-64. The 1000 subjects were characterized with respect to the following biomarkers and SNPs:

Biomarkers:

Total prostate-specific antigen (tPSA) [ng/mL]

Free prostate-specific antigen (fPSA) [ng/mL]

human kallikrein 2 (hK2) [ng/mL]

The ratio Free PSA/Total PSA (F/T PSA) was calculated and included in the data set.

SNPs:

rs12621278 (Chromosome 2, locus 2q31.1)

rs9364554 (Chromosome 6, locus 6q25.3)

rs10486567 (Chromosome 7, locus 7p15.2)

rs6465657 (Chromosome 7, locus 7q21.3)

rs2928679 (Chromosome 8, locus 8p21)

rs6983561 (Chromosome 8, locus 8q24.21)

rs16901979 (Chromosome 8, locus 8q24.21)

rs16902094 (Chromosome 8, locus 8q24.21)

rs12418451 (Chromosome 11, locus 11q13.2)

rs4430796 (Chromosome 17, locus 17q12)

rs11649743 (Chromosome 17, locus 17q12)

rs2735839 (Chromosome 19, locus 19q13.33)

rs9623117 (Chromosome 22, locus 22q13.1)

rs138213197 (Chromosome 17, locus 17q21)

rs1227732 (Chromosome 19, locus 19p13.11)

Background information for each subject was collected, including age and family history. Age was expressed in the units of years. Family history was graded in 4 levels, where 0 indicated no family history of PCa and 3 indicated extensive family history of PCa.

A first linear model was designed using only the information regarding the age of the subject, the family history and the F/T PSA. The first linear model is defined as:

EST1=1.07679−0.00118523*[AGE]+0.0952954*[FAMILYHISTORY]−0.0234183*[F/T PSA]

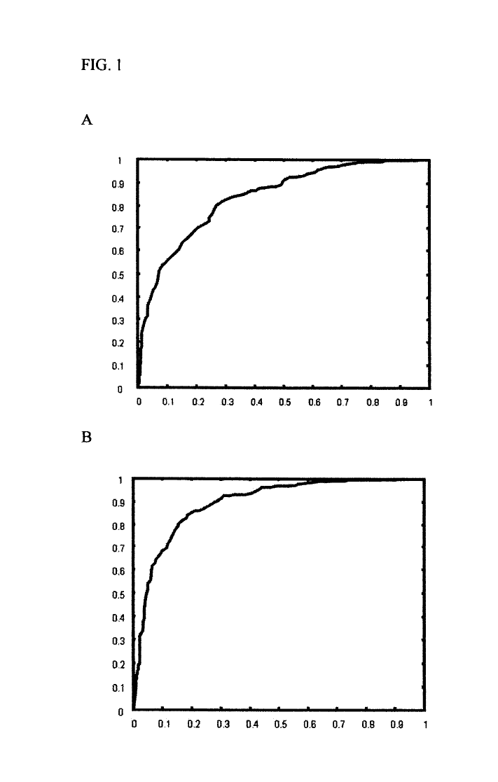

If EST1>0.5, the subject is likely to suffer from PCa. The diagnostic capability of the first linear model is, ROC-AUC=0.836, as illustrated in FIG. 1A.

A second linear model was designed, using all biomarkers available in this data set (i.e. age, family history, tPSA, fPSA, F/T PSA, and hK2). The second linear model is defined as:

EST2=0.806743−0.000112063*[AGE]+0.0541963*[FAMILYHISTORY]+0.000537*[tPSA]+0.0605211*[fPSA]−0.0218285*[F/TPSA]+0.624642*[hK2]

If EST2>0.5, the subject is likely to suffer from PCa. The diagnostic capability of the second linear model is, ROC-AUC=0.894, as illustrated in FIG. 1B.

In a third step, the impact of the genetic profile (i.e. the SNP data) was investigated. For example, in the current data set, the value for SNP rs12621278 was “AG” in only 83% of the cases (i.e. subjects known to suffer from PCa) compared to the controls (i.e. subjects known not to suffer from PCa), and hence the SNP rs12621278=“AG” is overrepresented in healthy individuals and is assigned a “SNP risk factor”=0.83. The value for SNP rs6983561 was “AC” in 142% of the cases in comparison to the controls, and hence the SNP rs6983561=“AC” is overrepresented in individuals suffering from PCa and is assigned a “SNP risk factor”=1.42. The SNP risk factor values for all selected SNPs are shown in Table 1.

TABLE 1

| SNP | Target value | SNP risk factor | Related to |
|---|---|---|---|
| rs12621278 | AG | 0.83 | PCa |
| rs9364554 | TT | 0.83 | PCa |
| rs10486567 | TT | 0.80 | PCa |
| rs6465657 | TT | 0.84 | PCa |
| rs2928679 | GG | 1.16 | PCa |
| rs6983561 | AC | 1.42 | PCa |
| rs16901979 | AC | 1.41 | PCa |
| rs16902094 | GG | 1.16 | PCa |
| rs12418451 | AA | 1.24 | PCa |
| rs4430796 | CC | 0.85 | PCa |
| rs11649743 | TT | 0.40 | PCa |
| rs2735839 | AA | 1.39 | PCa |
| rs9623117 | CC | 1.21 | PCa |
| rs138213197 | TC | 1.59 | PCa |
| rs1227732 | TT | 1.39 | MIC1 |

For an individual, the accumulative genetic score was calculated in the following approximate manner:

Start with an overall risk factor=1

For each SNP in Table 1, IF the subject matches the genetic criteria, multiply the overall risk factor with the SNP risk factor

Limit the cumulative overall risk factor to the interval [0.5,2] and scale the overall risk factor to match the output of the two linear models discussed above.

In algorithmic detail, the following defines the calculation of accumulative gene score:

```
f = 1
for each snp
  if subject matches snp criteria
    f = f * snp_risk_factor
  end
end
f = min( f , 2 )
f = max( f , 0.5 )
w = 0.05
if f<1
  snp_res = −w/f;
else
  snp_res = w*f;
end
```

The entity snp_res is scaled in the same manner as the output of the two linear models discussed above, and can be added to the model output to provide a better diagnostic tool than any of the models alone:

EST1g=EST1+snp_{—}res

and;

EST2g=EST2+snp_{—}res

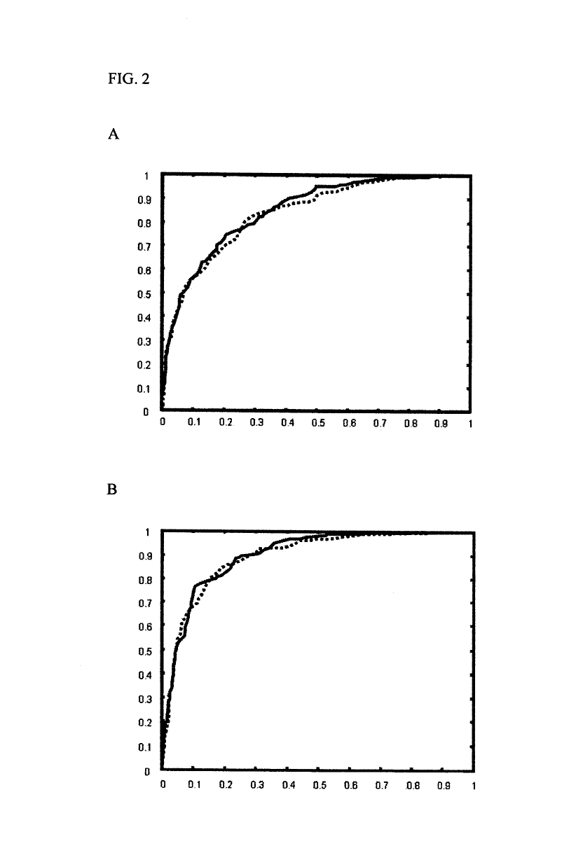

The ROC-AUC for EST1g was 0.846 and the ROC-AUC for EST2g was 0.899. The combination of genetic information and a linear model thus improves the diagnostic performance by 0.5-1% in terms of ROC-AUC, as illustrated in FIG. 2. FIG. 2A displays the ROC curve for the EST1 model (dotted line) and for the EST1g model (solid line). FIG. 2B displays the ROC curve for the EST2 model (dotted line) and for the EST2g model (solid line). Even though this may seem like a small number it may result in significant savings for the health care system. Considering that a large number of unnecessary biopsies are conducted based on the assessment of PSA alone, improving the diagnostic performance even by a small number will result in a clear benefit for both the health care providers in terms of lower cost and for the individual in terms of less suffering since an invasive diagnostic procedure could be avoided. Details of the clear clinical value of small ROC-AUC improvements have been discussed elsewhere (“Polygenic Risk Score Improves Prostate Cancer Risk Prediction: Results from the Stockholm-1 Cohort Study” by Markus Aly and co-authors as published in EUROPEAN UROLOGY 60 (2011) 21-28).

It is noteworthy that the control group was selected partly based on the total PSA value, meaning that there was known bias in the control group selection. This leads to an overestimated influence of the importance of the PSA related values. Thus, the diagnostic performance of the biomarker based models described in this example is overestimated. However, it is believed that the genetic profile suffers much less, or even not at all, from the PSA-bias in the control group. It is therefore assumed that the increase in diagnostic performance due to adding genetic marker information is true and accurate.

### Example 2

To illustrate the current invention even further, a data set comprising 417 cases (subjects known to suffer from PCa) and 396 controls (subjects known not to suffer from PCa) from the STHLM2 data set was extracted. The STHLM2 data set has been discussed in the public domain as evident on the web-page http://sthlm2.se/. In summary, during 2010-2012 about 26000 men who did a PSA test in the Stockholm area were included in the STHLM2 study. The 417+396=813 subjects were characterized with respect to the following biomarkers and SNPs:

Biomarkers:

Total prostate-specific antigen (tPSA) [ng/mL]

Intact prostate-specific antigen (iPSA) [ng/mL]

Free prostate-specific antigen (fPSA) [ng/mL]

human kallikrein 2 (hK2) [ng/mL]

Macrophage Inhibitory Cytokine 1 (MIC-1) [ng/mL]

beta-microseminoprotein (MSMB) [ng/mL]

SNPs:

657del5, rs10086908, rs1016343, rs10187424, rs1041449, rs10486567, rs1054564, rs10875943, rs10896449, rs10934853, rs10993994, rs11067228, rs11135910, rs11228565, rs11568818, rs11649743, rs11650494, rs11672691, rs11704416, rs12130132, rs12409639, rs12418451, rs12500426, rs12543663, rs12621278, rs12653946, rs1270884, rs130067, rs13252298, rs13385191, rs1354774, rs1363120, rs137853007, rs138213197, rs1447295, rs1465618, rs1512268, rs1571801, rs16901979, rs16902094, rs17021918, rs17632542, rs17879961, rs1859962, rs1894292, rs1933488, rs1983891, rs2018334, rs2121875, rs2242652, rs2273669, rs2292884, rs2405942, rs2660753, rs2735839, rs2736098, rs2928679, rs3213764, rs339331, rs3771570, rs3850699, rs3863641, rs401681, rs4245739, rs4430796, rs445114, rs4643253, rs4857841, rs4962416, rs5759167, rs5919432, rs5945619, rs6062509, rs620861, rs6465657, rs6763931, rs684232, rs6869841, rs6983267, rs6983561, rs7127900, rs7210100, rs721048, rs7241993, rs7611694, rs7679673, rs7931342, rs8008270, rs8102476, rs888663, rs902774, rs9364554, rs9600079, rs9623117

Background information for each subject was collected, including if the subject had undergone a previous biopsy of the prostate, age and family history (yes or no). Age was expressed in the units of years.

In order to decide which subjects that should be referred to biopsy, it is required to predict a value for each tested subject that is correlated with the probability that said subject has prostate cancer. This can be done by combining measured values of the biomarkers and other information in the following equation:

y=0.0275109+0.4272770*prevBiop+0.0006496*tPSA+0.0868130*score−0.0334401*hk2+0.0082864*iPSA+0.0110069*mic1+0.0069329*msmb+0.0084636*age−0.0018337*fPSA−1.6079442*(fPSA/tPSA)

In this equation, ‘prevBiops’ indicates if the subject has been biopsied before (1) or not (0), ‘score’ is the genetic score variable computed as described in the public report “Polygenic Risk Score Improves Prostate Cancer Risk Prediction: Results from the Stockholm-1 Cohort Study” by Markus Aly and co-authors as published in EUROPEAN UROLOGY 60 (2011) 21-28, containing the validated prostate cancer susceptibility SNPs (said SNP being related to cancer susceptibility or related to PSA, free-PSA, MSMB and/or MIC-1 biomarker plasma levels) listed in the present example. The parameters ‘HK2’, ‘fPSA’, ‘iPSA’, ‘MIC1’, ‘MSMB’, ‘tPSA’ refers to the respective measured values of these biomarkers and ‘age’ is the age of the subject. The equation was derived using the ordinary least squares estimator (other linear estimators can also straight-forwardly be used, e.g. the logistic regression estimator) with untransformed parameters. In this particular model, information regarding family history was omitted.

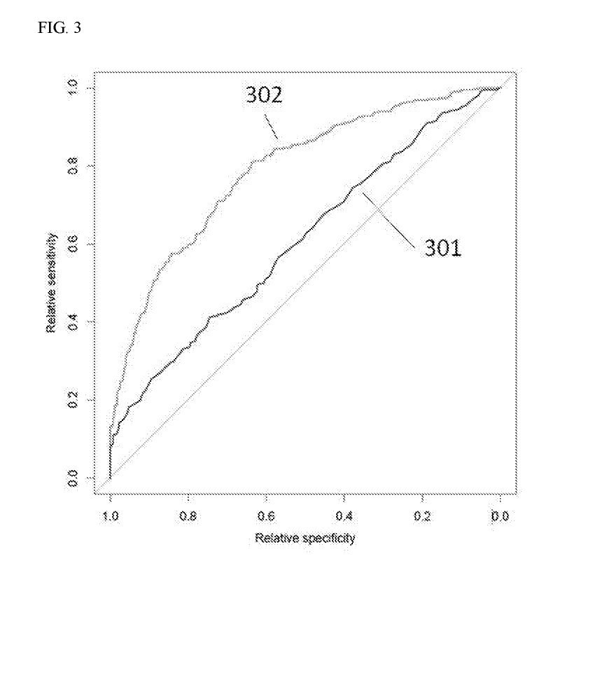

The resulting value ‘y’ will be strongly correlated with the risk of having prostate cancer, as illustrated in FIG. 3. The ROC curves in FIG. 3 represent PSA (301) alone and the model described in this example (302). If y is above a cutoff value the man should be recommended a referral to an urologist for examination of the prostate using biopsies.

The value of the cutoff depends on the tradeoff between test sensitivity and specificity. If, for example, the cut off value of 0.44 is used, this particular test will result in test sensitivity of 0.8 and specificity of 0.54. This can be compared to using the PSA value alone as a screening test, which results in a sensitivity of 0.8 and specificity of 0.30. In practice, this means that this particular model as applied to the population of 813 subjects would result in the same number of detected prostate cancers as the PSA test, but with 95 subjects less being referred to biopsy, which corresponds to an improvement of approximately 15% compared to the PSA test alone. If, as a second example, the cut off value of 0.37 is used, this particular test will result in test sensitivity of 0.9 and specificity of 0.32. At the sensitivity level 0.9, approximately 7% of the biopsies as predicted using PSA would be saved.

### Example 3

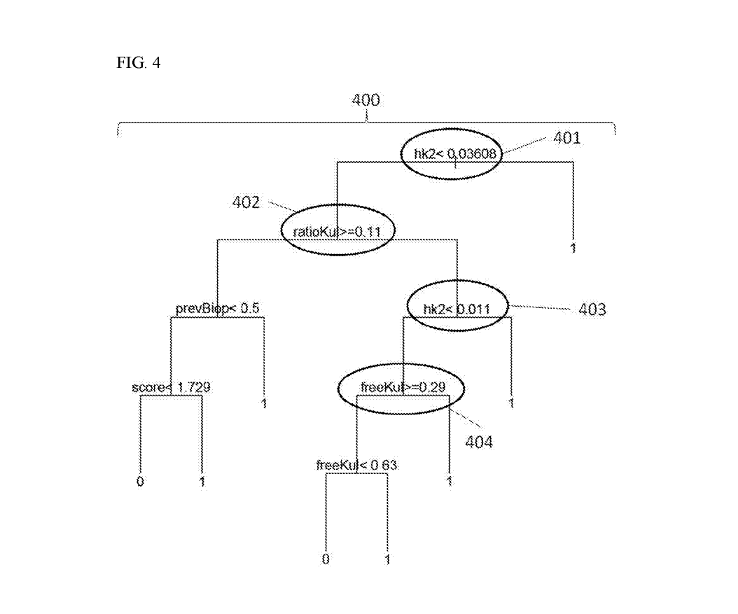

To illustrate the current invention even further, an alternative computational method for obtaining a prediction was applied. Equations such as those presented in Examples 1 and 2 are not the only way in which the biomarkers can be combined to predict PCa. In fact, the method for calculating y in order to predict PCa can be intricate and not even possible to write down on a sheet of paper. A more complicated but very powerful example of how the biomarkers can be combined is to use a forest of decision trees. An example of a decision tree (400) is depicted in FIG. 4. Suppose that a subject tested for biomarkers and SNPs with results HK2=0.009, fPSA=0.20, tPSA=2.53. When applying the decision tree (400) as exemplified in FIG. 4, the top node (401) is related to the hk2 value. Since the subject has a HK2 value which satisfies the node condition, one follows the left branch from that node. The second node (402) is also related to the ratio of fPSA and tPSA. The subject has a ratio of 0.20/2.53=0.079 which does not satisfy the node condition; then one follows the right branch from that node. The third level node (403) is related to hk2. Since the subject hk2 value does satisfy the node condition, one follows the left branch from that node. The fourth level node (404) is related to the fPSA value, and since the fPSA value of the subject does not satisfy the node condition, one follows the right branch from that node. At this point, there are no more nodes meaning that one has reached a leaf of the decision tree. Each leaf has a corresponding output; in this particular example, a leaf value of “1” means “do refer to biopsy” and “0” means “do not refer to biopsy”. The exemplary subject did in this case end up in a leaf with value “1”, meaning that the prediction provided by this decision tree is “yes: do refer to biopsy”.

A problem with relying on merely one decision tree for calculating y to predict PCa is that a single decision tree has very high variance (i.e. if the data changed slightly the calculated value of y is also likely to change, leading to variance in the prediction of PCa), although its bias is very low. One possible method for reducing the high variance is to construct a forest of decorrelated trees using the random forest algorithm as described in the report “Random Forests” by Leo Breiman as published in Machine Learning 45 (1): 5-32 (2001) (which is incorporated by reference herein). A large number of trees are grown, and before the growth of each tree the data is randomly perturbed in such a way that the expected value of its prediction is unchanged. To predict PCa, all trees cast a vote to decide whether a subject should be referred to biopsy. Such a voting prediction retains the unbiased properties of decision trees, however considerably lowers the variance (similarly to how the variance of a mean is lower than the variance of the individual measurements used to compute the mean). Since the random forest algorithm depends on random number generation, it is impossible to write down the resulting prediction algorithm in closed form.

When applied to the data set as described in Example 2, this model can at sensitivity 0.8 save approximately 21% of the number of biopsies compared to PSA alone. At sensitivity 0.9, approximately 13% of the number of biopsies would be saved compared to using PSA alone.

### Example 4

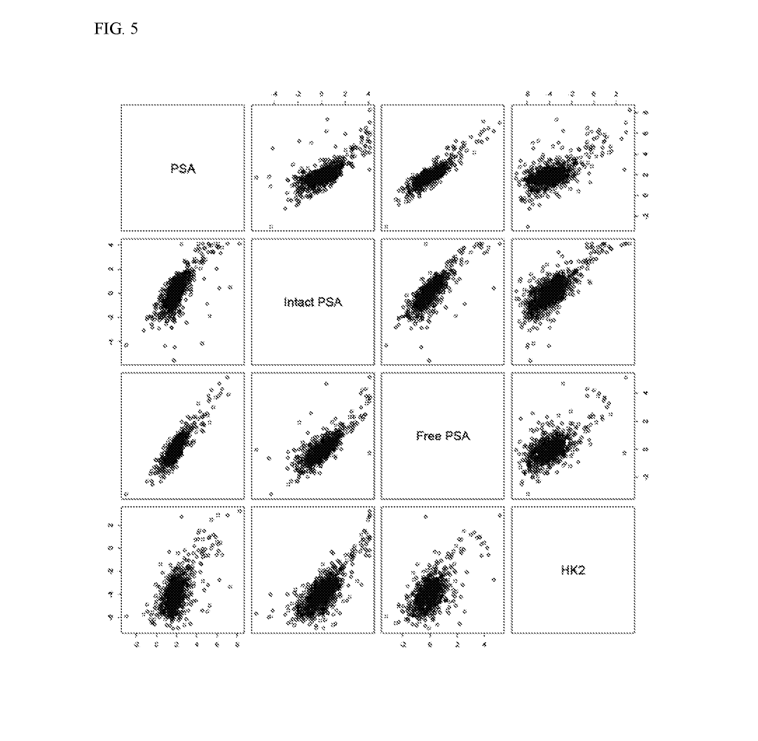

To illustrate the redundancy aspect of the invention, values for the protein biomarkers total PSA (tPSA), intact PSA (iPSA), free PSA (fPSA), and HK2 were plotted pair wise versus each other (after logarithm transformation), as shown in FIG. 5. The values were obtained from 450 individuals of whom 216 were confirmed having PCa, according to a part of the study STHLM2 study described in previous examples. The values were further mean centered and scaled to unit variance prior to creating the plots. Since all these pair wise plots display an overall correlation, it is foreseeable that if any one of these four protein biomarker values was omitted, the remaining three would in concert provide essentially the same information. To verify this, a kallikrein score K was defined as:

K=(0.07316*tPSA−0.13778*fPSA+0.01293*HK2+0.08323*iPSA−0.01844*f/tPSA)/(0.07316−0.13778+0.01293+0.08323−0.01844)

In this equation the parameters ‘HK2’, ‘iPSA’, ‘tPSA’, ‘fPSA’ and ‘f/tPSA’ refers to the respective measured values in ng/mL of these biomarkers. Biomarker values were applied without transformation, i.e. in original units. The definition of K is made in a manner that any one of the contributing terms can be removed. If for some reason the HK2 value is missing for a particular individual, K would be estimated as:

K′=(0.07316*tPSA−0.13778*fPSA+0.08323*iPSA−0.01844*f/tPSA)/(0.07316−0.13778+0.08323−0.01844)

If for some reason the HK2 value and the iPSA value are missing for a particular individual, K would be estimated as:

K″=(0.07316*tPSA−0.13778*fPSA−0.01844*f/tPSA)/(0.07316−0.13778−0.01844)

If for some reason the HK2 value and the iPSA value are missing and the quotient F/T PSA is not calculated for a particular individual, K would be estimated as:

K′″=(0.07316*tPSA−0.13778*fPSA)/(0.07316−0.13778)

Next, a predictive model for assessing PCa risk was designed using the kallikrein score as the information carrier regarding the kallikrein biomarkers:

Y=K*C1+score*C2+MIC1*C3+MSMB*C4+age*C5+C6

wherein Y is the risk for PCa, ‘score’ is the genetic score variable computed as described in previous examples, ‘MIC1’ and ‘MSMB’ refers to the respective measured values in ng/mL of these biomarkers, and age refers to the age of the individual. C1-C6 are constants adjusting for the contribution of each component. This model for assessing PCa risk was applied using the kallikrein score K as calculated using the full set of biomarkers (K) or using the estimated K with a reduced number of protein biomarkers (K′, K″, and K′″). The performance of the risk assessment was estimated using ROC-AUC.

For the case of using all available kallikrein biomarkers for calculating K, the ROC-AUC value was 0.77. When instead using K′ as an estimate of K in the risk assessment model, meaning that all HK2 values were ignored, the result was also a ROC-AUC value of 0.77. When using K″ (i.e. ignoring HK2 and iPSA values) the ROC-AUC value was 0.74 and for K′″ the ROC-AUC value was also 0.74.

Since the difference in performance between the results obtained using K, K′, K″, or K′″ in the risk assessment model is small, it is concluded that for the protein biomarkers in this example, it is possible to omit one or a few values and still get a similar performance as the full model. This makes it possible to assess the risk for individuals where one or a few biomarker values are missing.

In order to illustrate that the genetic score comprises a similar robustness to missing information, the available genetic information for individuals was reduced by 5%. In practice, for any given individual, values for a few genetic markers will typically be missing due to difficulties in the assay for detecting said genetic information. Hence, in the examples above, for each individual there is information available for about 98% of the SNP listed for inclusion in the model. To test how the model performs under a larger loss of genetic information, another 5% of the SNP information was randomly removed from the individuals in the present example, and the full model and the K′ model were re-evaluated. In the case of the full model, the genetics-depreciated score produced a ROC-AUC of 0.75 (to compare with 0.77 for the non-genetics-depreciated model) and for the K′ model the genetics-depreciated score produced a ROC-AUC of 0.73 (to compare with 0.77 for the non-genetics-depreciated K′ model).

The same procedure can be applied to any other combinations of the biomarkers contributing to the kallikrein score. Furthermore, other biomarkers with redundancy can be grouped together to produce a summary score, in a similar manner as described for kallikrein biomarkers.

In summary, this example illustrates that the method is robust to small alterations of input variables. The loss of one or a few biomarkers or a few % of the genetic information does still result in acceptable performance, albeit with slightly reduced performance. The wide applicability of the present model is important from a socioeconomic perspective, where also individuals, for whom technical issues may have resulted in minor loss of information, can get their risk for PCa assessed.

### Example 5

To illustrate the contribution of SNP related to biomarker level to the performance of the model of the present invention, a predictive linear model was made using the following parameters: previous biopsies, tPSA, score, HK2, fPSA, iPSA, MIC1, MSMB, age, f/tPSA, and tPSA/psaScore. The data set applied was the same as described in Example 4. The parameter tPSA/psaScore refers to SNP information related to the total PSA value, and the remaining parameters are as defined in previous examples. Two models were made, one including tPSA/psaScore and one excluding tPSA/psaScore. The model including tPSA/psaScore had a ROC-AUC of 0.77 as estimated using a cross-validation procedure. The model excluding tPSA/psaScore had a ROC-AUC of 0.74 using the same procedure. This shows that genetic information which is related to biomarker level can have positive impact on the performance of a PCa predictive model.

Although the invention has been described with regard to its preferred embodiment, which constitutes the best mode currently known to the inventor, it should be understood that various changes and modifications as would be obvious to one having ordinary skill in this art may be made without departing from the scope of the invention as set forth in the claims appended hereto.

1. A method for indicating a presence or non-presence of prostate cancer (PCa) in an individual, comprising the steps of:
1. Providing at least one biological sample from said individual;
2. In said biological sample, determining
a) a presence or concentration of at least one PCa related biomarker;
b) a PCa related genetic status of said individual by determining a presence of at least one SNP related to PCa; and
c) a PCa biomarker concentration related genetic status of said individual by determining a presence of at least one SNP related to a PCa biomarker concentration;
3. Combining data from said individual regarding said presence or concentration of at least one PCa related biomarker, data from said individual regarding said PCa related genetic status, and data from said individual regarding PCa biomarker concentration related genetic status, to form an overall composite value;
4. Correlating said overall composite value to the presence of PCa in said individual by comparing the overall composite value to a pre-determined cut-off value established with control samples of known PCa and benign disease diagnosis;
wherein the presence or concentration of at least one of the biomarkers (i) PSA, (ii) total PSA (tPSA), (iii) intact PSA (iPSA), (iv) free PSA (fPSA), and (v) HK2, is determined and included in the overall composite value.

2. A method for indicating a presence or non-presence of prostate cancer (PCa) in an individual, comprising the steps of:
1. Providing at least one biological sample from said individual;
2. In said biological sample, determining
a) a presence or concentration of at least two PCa related biomarkers; and
b) a PCa related genetic status of said individual by determining a presence of at least one SNP related to PCa;
3. Combining data from said individual regarding said presence or concentration of at least two PCa related biomarkers, to form a biomarker composite value representing the PCa biomarker-related risk of developing PCa;
4. Combining data from said individual regarding said PCa related genetic status, to form a genetics composite value representing the genetics-related risk of developing PCa;
5. Combining the biomarker composite value and the genetics composite value to form an overall composite value to predict the presence of PCa in said individual by comparing said overall composite value to a pre-determined cut-off value established with control samples of known PCa and benign disease diagnosis;
wherein the presence or concentration of at least one and at most four of the biomarkers (i) PSA, (ii) total PSA (tPSA), (iii) intact PSA (iPSA), (iv) free PSA (fPSA), and (v) HK2, is determined and included in the biomarker composite value.

3. The method of claim 2, further comprising a step 2 c) determining, in said biological sample a PCa biomarker concentration related genetic status of said individual by determining a presence of at least one SNP related to a PCa biomarker concentration;
and step 4 comprises combining data from said individual regarding said PCa related genetic status and said PCa biomarker concentration related genetic status, to form a genetics composite value representing the genetics-related risk of developing PCa.

4. The method of claim 1, wherein the SNP related to PCa includes at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197.

5. The method of claim 1, wherein the SNP related to a PCa biomarker concentration includes at least one of rs3213764, rs1354774, and rs1227732.

6. The method of claim 1, further comprising determining a Body Mass Index (BMI) related genetic status of said individual by determining a presence of at least one SNP related to the BMI, and wherein data from said individual regarding said SNP related to the BMI are included in the combined data forming said composite value.

7. The method of claim 6, wherein the SNP related to the BMI of said individual includes at least one of rs3817334, rs10767664, rs2241423, rs7359397, rs7190603, rs571312, rs29941, rs2287019, rs2815752, rs713586, rs2867125, rs9816226, rs10938397, and rs1558902.

8. The method of claim 1, further comprising collecting the family history regarding PCa and physical data from said individual, and wherein said family history and physical data are included in the combined data forming said overall composite value.

9. The method of claim 1, wherein the presence or concentration of MIC-1 and/or MSMB is further determined and included either in the biomarker composite value or directly in the overall composite value.

10. The method of claim 1, wherein said biological sample is a blood sample.

11. The method of claim 1, wherein said overall composite value and/or said biomarker composite value and/or said genetics composite value is calculated using a method in which the non-additive effect of a SNP related to a PCa biomarker concentration and the corresponding PCa biomarker concentration is utilized.

12. The method of claim 1, wherein the determination of the genetic status is conducted by use of MALDI mass spectrometry.

13. The method of claim 1, wherein the determination of a presence or concentration of said PCa biomarkers is conducted by use of microarray technology.

14. An assay device comprising a solid phase having immobilised thereon at least three different types of ligands, wherein:
the first type of said ligands comprises at least one ligand, which binds specifically to a biomarker related to PCa, such as at least one of PSA, iPSA, tPSA, fPSA, and hK2, and optionally also MIC-1 and/or MSMB;
the second type of said ligands comprises at least one ligand, which binds specifically to a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197; and
the third type of said ligands comprises at least one ligand, which binds specifically to a SNP related to a PCa biomarker concentration, such as at least one of rs3213764, rs1354774 and rs1227732.

15. An assay device, comprising a solid phase having immobilised thereon at least two different types of ligands, wherein:
the first type of said ligands comprises at least two different ligands, each of which binding specifically to a biomarker related to PCa, such as at least one of PSA, iPSA, tPSA, fPSA, and hK2, and optionally also MIC-1 and/or MSMB; and
the second type of said ligands comprises at least one ligand, which binds specifically to a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197.

16. The assay device of claim 15, wherein the solid phase further has a third type of ligand immobilised, wherein said third type of ligand comprises at least one ligand, which binds specifically to a SNP related to a PCa biomarker concentration, such as at least one of rs3213764, rs1354774 and rs1227732.

17. The assay device of claim 14, wherein the solid phase further has a fourth type of ligand immobilised, wherein said fourth type of ligand comprises at least one ligand, which binds specifically to a SNP related to the BMI, such as at least one of rs3817334, rs10767664, rs2241423, rs7359397, rs7190603, rs571312, rs29941, rs2287019, rs2815752, rs713586, rs2867125, rs9816226, rs10938397, and rs1558902.

18. A test kit, comprising an assay device according to claim 14 and at least three different types of detection molecules, wherein:
the first type of said detection molecules comprises at least one ligand, which is capable of detecting a biomarker related to PCa, such as at least one of PSA, iPSA, tPSA, fPSA, and hK2, and optionally also MIC-1 and/or MSMB;
the second type of said detection molecules comprises at least one ligand, which is capable of detecting a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197; and
the third type of said detection molecules comprises at least one ligand, which is capable of detecting a SNP related to a PCa biomarker concentration, such as at least one of rs3213764, rs1354774 and rs1227732.

19. A test kit, comprising an assay device according to claim 15 and at least two different types of detection molecules, wherein:
the first type of said detection molecules comprises at least two different detection molecules, each of which is capable of detecting a biomarker related to PCa, such as at least one of PSA, iPSA, tPSA, fPSA, and hK2, and optionally also MIC-1 and/or MSMB, provided that said at least two different detection molecules are capable of detecting different biomarkers related to PCa; and
the second type of said detection molecules comprises at least one ligand, which is capable of detecting a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197.

20. A test kit 3, comprising an assay device according to claim 16 and at least three different types of detection molecules, wherein:
the first type of said detection molecules comprises at least two different detection molecules, each of which is capable of detecting a biomarker related to PCa, such as at least one of PSA, iPSA, tPSA, fPSA, and hK2, and optionally also MIC-1 and/or MSMB, provided that said at least two different detection molecules are capable of detecting different biomarkers related to PCa;
the second type of said detection molecules comprises at least one ligand, which is capable of detecting a SNP related to PCa, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117 and rs138213197; and
the third type of said detection molecules comprises at least one ligand, which is capable of detecting a SNP related to a PCa biomarker concentration, such as at least one of rs3213764, rs1354774 and rs1227732.

21. The test kit of claim 18, wherein the assay device solid phase further has a fourth type of ligand immobilised, wherein said fourth type of ligand comprises at least one ligand which binds specifically to a SNP related to the BMI, the test kit further comprising a fourth type of detection molecule, wherein the fourth type of ligand comprises at least one ligand, which is capable of detecting a SNP related to the BMI, such as at least one of rs3817334, rs10767664, rs2241423, rs7359397, rs7190603, rs571312, rs29941, rs2287019, rs2815752, rs713586, rs2867125, rs9816226, rs10938397, and rs1558902.

22.-25. (canceled)

26. A non-transitory computer-readable medium comprising instructions for causing a computer to perform at least steps 3 and 4 of the method of claim 1; such as steps 1, 2a, 2b, 2c, 3 and 4 of the method of claim 1.

27. A non-transitory computer-readable medium comprising instructions for causing a computer to perform at least steps 3 and 4 of the method of claim 2; such as steps 1, 2a, 2b, 3 and 4 of the method of claim 2; and optionally further comprising instructions for causing a computer to perform step 5 of the method of claim 2.

28. A non-transitory computer readable medium, comprising instructions for causing a computer to perform at least steps 2c, 3 and 4 of the method of claim 3.

29. The computer readable medium of claim 26, further comprising instructions for causing a computer to perform a method comprising determining a Body Mass Index (BMI) related genetic status of said individual by determining a presence of at least one SNP related to the BMI, and wherein data from said individual regarding said SNP related to the BMI are included in the combined data forming said composite value.

30. An apparatus comprising an assay device according to claim 22 and a non-transitory computer readable medium comprising instructions for causing a computer to perform the following steps: combining data from an individual regarding a presence or concentration of at least one PCa related biomarker, data from said individual regarding said PCa related genetic status, and data from said individual regarding PCa biomarker concentration related genetic status, to form an overall composite value, and correlating said overall composite value to the presence of PCa in said individual by comparing the overall composite value to a pre-determined cut-off value established with control samples of known PCa and benign disease diagnosis.
